(19) United States
(12) Patent Application Publication
(10) Pub. No.: US 2025/0339673 A1
(43) Pub. Date: Nov. 6, 2025

(54) ELECTROTHERAPY DEVICE

(71) Applicant: OMRON HEALTHCARE Co., Ltd., Kyoto (JP)

(72) Inventor: Tomoyuki NISHIDA, Kyoto (JP)

(21) Appl. No.: 19/271,134

(22) Filed: Jul. 16, 2025

Related U.S. Application Data
(63) Continuation of application No. PCT/JP2023/040551, filed on Nov. 10, 2023.

(30) Foreign Application Priority Data
Mar. 14, 2023 (JP) 2023-039605

(51) Int. Cl. A61N 1/04

(52) U.S. Cl. CPC A61N 1/0476

(57) ABSTRACT

An electrical treatment device includes a tubular member, a main body portion fixed to the tubular member, and an electrode portion provided at the tubular member such that the electrode portion is attachable to a body surface of a user. The tubular member includes a first portion located on the body surface side of the user in a usage state and a second portion located on a side opposite to the body surface side of the user. The first portion includes a first surface that faces the body surface side of the user in a usage state. The electrode portion is provided at the first surface. The main body portion is fixed to the second portion such that at least a part of the main body portion overlaps the second portion.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040551, filed Nov. 10, 2023, which application claims priority to Japanese Patent Application No. 2023-039605, filed Mar. 14, 2023, which applications are incorporated herein by reference in their entireties.

### TECHNICAL FIELD

The present disclosure relates to an electrical treatment device.

### BACKGROUND ART

As a known electrical treatment device, JP 2007-44479A (Patent Document 1) discloses a low-frequency treatment device including a flattened bag filled with inorganic beads, in which a skin-contact electrode portion composed of a pair of conductive fabrics and a terminal portion for supplying a low-frequency current to the electrode portion are integrally provided on one surface of the bag.

In addition, JP 2017-189268A (Patent Document 2) discloses an electrical treatment device including a pair of conductive adhesive sheets linearly arranged with a space therebetween, and a treatment device main body (main body portion) provided so as to straddle the pair of conductive adhesive sheets, in which a clip is provided on one of the pair of conductive adhesive sheets.

### CITATIONS

Patent Document 1: JP 2007-44479A
Patent Document 2: JP 2017-189268A

### SUMMARY

### Technical Problem

In the electrical treatment device disclosed in Patent Document 1, in use, the weight of the beads filled in the bag presses the skin-contact electrode portion against the body surface of the user. As such, the placement of the electrical treatment device is limited, such as installation so as to straddle the shoulders in the front-back direction. In addition, depending on the placement location of the electrical treatment device, it may be difficult to maintain stable adhesion of the device to the body surface.

In the electrical treatment device disclosed in Patent Document 2, a clip is used to sequentially peel off, starting from the end portion of one of the pair of conductive adhesive sheets, the one conductive adhesive sheet, the main body portion, and the other conductive adhesive sheet. As a result, it is difficult to detach the electrical treatment device entirely from the treatment site. Further, when the electrical treatment device is placed on the back or inside clothing and the clip is not visible, it may be difficult to locate the clip, thus making detachment troublesome.

In view of the above-described problems, an object of the present disclosure is to provide an electrical treatment device that can be attached to the body surface and can be easily detached from the body surface, thereby offering high convenience.

### Solution to Problem

An electrical treatment device according to the present disclosure includes: a tubular member, and an electrode portion provided at the tubular member such that the electrode portion is attachable to a body surface of a user. The tubular member includes a first portion located on the body surface side of the user in a usage state and a second portion located on a side opposite to the body surface side of the user. The first portion includes a first surface that faces the body surface side of the user in a usage state. The electrode portion is provided at the first surface.

According to the above-mentioned configuration, the electrical treatment device can be attached to the body surface by means of the electrode portion. In addition, when detaching the electrical treatment device, the second portion located on the side opposite to the first portion side on which the electrode portion is provided can be easily grabbed, and the tubular member can be easily grasped by inserting a finger or the like to the inside of the tubular member. By moving the tubular member in a direction away from the body surface, the peeling force can be applied toward the center side of the electrode portion from both end sides of the electrode portion in the circumferential direction. As a result, the electrical treatment device can be easily detached, thereby improving convenience.

The electrical treatment device according to the present disclosure may include a main body portion fixed to the tubular member. The main body portion may be fixed to the second portion such that at least a part of the main body portion overlaps the second portion.

According to the above-mentioned configuration, by grabbing the main body portion and moving it in the direction away from the body surface, the peeling force can be applied to the electrode portion, and the electrical treatment device can be entirely and easily detached from the body surface.

In the electrical treatment device according to the present disclosure, the tubular member may include a first tubular member and a second tubular member disposed with a space between the first tubular member and the second tubular member. Each of the first tubular member and the second tubular member includes the first portion and the second portion. The electrode portion may include a first electrode portion provided at the first surface provided in the first tubular member, and a second electrode portion provided at the first surface provided in the second tubular member. In this case, the main body portion may be fixed to the second portion provided in the first tubular member and the second portion provided in the second tubular member so as to straddle the first tubular member and the second tubular member.

According to the above-mentioned configuration, the main body portion is fixed to the first tubular member and the second tubular member so as to straddle the first tubular member and the second tubular member disposed with a space therebetween. In this manner, by grabbing the main body portion and moving it in the direction away from the body surface, the peeling force can be simultaneously applied to the first electrode portion and the second electrode portion, and the electrical treatment device can be entirely and easily detached from the body surface.

In the electrical treatment device according to the present disclosure, a first tubular axis provided in the first tubular member and a second tubular axis provided in the second tubular member may be parallel to each other. The first tubular axis and the second tubular axis may be orthogonal to a direction in which the first tubular member and the second tubular member are disposed side by side in plan view.

According to the above-mentioned configuration, by grabbing the main body portion and moving it in the direction away from the body surface, for each of the first electrode portion provided in the first tubular member and the second electrode portion provided in the second tubular member, the peeling force toward the inside of the first tubular member and the second tubular member in the circumferential direction can be applied to both end portions of the first electrode portion and the second electrode portion in the arrangement direction of the first tubular member and the second tubular member. In this manner, the main body portion, the first tubular member, and the second tubular member can be entirely and easily detached from the body surface.

In the electrical treatment device according to the present disclosure, the tubular member may include a first tubular member and a second tubular member disposed with a space between the first tubular member and the second tubular member. Each of the first tubular member and the second tubular member includes the first portion and the second portion. The electrode portion may include a first electrode portion provided at the first surface provided in the first tubular member, and a second electrode portion provided at the first surface provided in the second tubular member. A fixing member fixed to the main body portion may be further provided. The fixing member may include a first fixing portion fixed to the second portion provided in the first tubular member, and a second fixing portion fixed to the second portion provided in the second tubular member. In this case, one end side of the main body portion in an arrangement direction in which the first tubular member and the second tubular member are disposed side by side may be fixed to the first fixing portion, and another end side of the main body portion in the arrangement direction is fixed to the second fixing portion, and thus the main body portion may be fixed to the first tubular member and the second tubular member through the fixing member.

According to the above-mentioned configuration, the main body portion is fixed so as to straddle the first fixing portion fixed to the first tubular member and the second fixing portion fixed to the second tubular member. In this manner, by grabbing the main body portion and moving it in the direction away from the body surface, the peeling force can be simultaneously applied to the first electrode portion and the second electrode portion, and the electrical treatment device can be entirely and easily detached from the body surface.

In the electrical treatment device according to the present disclosure, a first tubular axis provided in the first tubular member and a second tubular axis provided in the second tubular member may be parallel to the arrangement direction in plan view.

According to the above-mentioned configuration, by grabbing the main body portion and moving it in the direction away from the body surface, for each of the first electrode portion provided in the first tubular member and the second electrode portion provided in the second tubular member, the peeling force toward the inside of the first tubular member and the second tubular member in the circumferential direction can be applied to both end portions of the first electrode portion and the second electrode portion in the tube axial direction. In this manner, the main body portion, the first tubular member, and the second tubular member can be entirely and easily detached from the body surface.

In the electrical treatment device according to the present disclosure, a length of the first portion in a direction parallel to a tube axial direction of the tubular member may be constant in a circumferential direction. The second portion may include a portion where a length in the direction parallel to the tube axial direction is smaller than that of the first portion.

With the second portion having the above-described shape, the area of the tubular member can be reduced, and the weight and manufacturing cost of the electrical treatment device can be reduced.

### Advantageous Effects of Invention

According to the present disclosure, it is possible to provide an electrical treatment device that can be attached to the body surface and can be easily detached from the body surface, thereby offering high convenience.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic view of an electrical treatment device according to Embodiment 1;

FIG. 2 is a schematic view of an electrical treatment device according to Embodiment 2;

FIG. 3 is a diagram illustrating a state of detaching an electrical treatment device according to Embodiment 2;

FIG. 4 is a schematic view of an electrical treatment device according to Embodiment 3;

FIG. 5 is a schematic view of an electrical treatment device according to a first modified example; and,

FIG. 6 is a schematic view of an electrical treatment device according to Embodiment 4.

### DESCRIPTION OF EMBODIMENTS

Embodiments of the present disclosure are described below with reference to the drawings. In the embodiments described below, identical or corresponding components are denoted by the same reference numerals in the drawings, and repeated descriptions thereof are omitted.

In a non-limiting manner and with reference to the drawings, the present disclosure describes an electrical treatment device that can be attached to a body surface and can be easily detached from the body surface, thereby offering high convenience. In one possible embodiment an electrical treatment device 100A includes a tubular member 20, a main body portion 10 fixed to the tubular member 20, and an electrode portion 30 provided at the tubular member 20 such that the electrode portion is attachable to a body surface of a user. The tubular member 20 includes a first portion 21 located on the body surface side of the user in a usage state and a second portion 22 located on a side opposite to the body surface side of the user. The first portion 21 includes a first surface that faces the body surface side of the user in a usage state. The electrode portion 30 is provided at the first surface. The main body portion 10 is fixed to the second portion 22 such that at least a part of the main body portion overlaps the second portion 22.

Adverting now to the various contemplated embodiments of the present disclosure.

### Embodiment 1

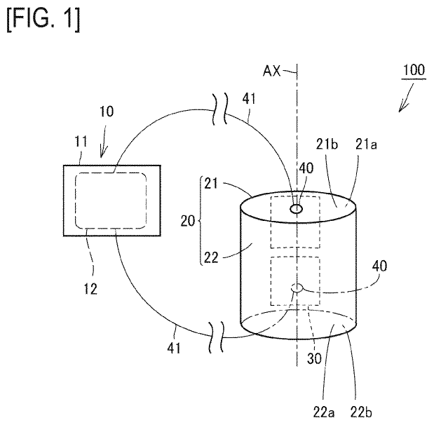

FIG. 1 is a schematic view of an electrical treatment device according to Embodiment 1. With reference to FIG. 1, an electrical treatment device 100 according to Embodiment 1 is described.

As illustrated in FIG. 1, the electrical treatment device 100 according to Embodiment 1 includes a main body portion 10, a tubular member 20, two electrode portions 30, two terminal portions 40, and two wiring portions 41.

The electrical treatment device 100 is, for example, a low-frequency treatment device that alleviates a user's pain by supplying a low-frequency pulsed current to the electrode portion 30 in contact with the body surface of the user. The frequency of low-frequency pulsed current is, for example, 1 Hz to 1200 Hz.

The main body portion 10 is disposed at a position separated from the tubular member 20. The main body portion 10 includes a housing case 11 and a circuit portion 12. The housing case 11 has a substantially box shape, and accommodates the circuit portion 12 inside. The circuit portion 12 is electrically connected to the electrode portion 30 through the wiring portion 41 and the terminal portion 40. The circuit portion 12 controls the current, and voltage and the like flowing through the electrode portion 30. The circuit portion 12 is a circuit board on which a desired circuit is formed.

The tubular member 20 is formed in a tubular shape, and has a tubular axis AX. The tubular member 20 is formed in a circular tubular shape, for example. Note that, the shape of the tubular member 20 is not limited to the circular tubular shape as long as it is formed in a tubular shape continuously in the circumferential direction while ensuring the region for providing the electrode portion 30.

The tubular member 20 includes a first portion 21 that is located on the body surface side of the user in a usage state, and a second portion 22 that is located on the side opposite to the body surface side of the user in a usage state. The tubular member 20 is formed with the first portion 21 and the second portion 22 that are continuous in the circumferential direction.

The first portion 21 includes an outer surface 21a and an inner surface 21b. The outer surface 21a corresponds to a first surface that faces the body surface side of the user in a usage state. The second portion 22 includes an outer surface 22a and the inner surface 22b. The above-mentioned outer surface 21a and outer surface 22a are continuous in the circumferential direction, and form the outer surface of the tubular member 20. The inner surface 21b and inner surface 22b are continuous in the circumferential direction, and form the inner surface of the tubular member 20.

Each of the two electrode portions 30 is provided on the tubular member 20 such that it is attachable to the body surface of the user. The two electrode portions 30 are disposed side by side in a direction parallel to the tubular axis AX. The electrode portion 30 may be, for example, a gel pad, or may be a structure including both an adhesive layer and an electrode.

The electrode portion 30 is provided at the outer surface 21a of the above-mentioned first portion 21. The electrode portion 30 has a substantially rectangular shape. To be more specific, the electrode portion 30 has a substantially rectangular shape in which the pair of sides facing each other in the circumferential direction is approximately parallel to the tubular axis AX direction. Note that, the shape of the electrode portion 30 is not limited to a rectangular shape, and may be of any appropriate shape such as a circular shape, an elliptical shape, a polygonal shape, or an oval shape. In addition, the line making up the outer edge of the electrode portion is not limited to the straight line, and may be zigzag or curved.

The two terminal portions 40 are provided corresponding to the above-mentioned two electrode portions 30. The two terminal portions 40 are provided on the inner surface 21b side of the above-mentioned first portion 21. In this manner, the terminal portion 40 can be provided at a position close to the electrode portion 30. The wiring portion 41 can be detachably mounted to the terminal portion 40. Note that, while the present embodiment describes an exemplary case where the two wiring portions 41 are connected to the corresponding two terminal portions 40, this is not limitative. A single wiring portion 41 may be configured to branch into two on the terminal portion 40 side, with each of the branched portions being connected to the corresponding terminal portion 40.

In the present embodiment, the electrical treatment device 100 can be attached to the body surface by means of the electrode portion 30. In addition, when detaching the electrical treatment device 100, it is easy to grab the second portion 22 located on the side opposite to the first portion 21 side on which the electrode portion 30 is provided, or grasp the tubular member 20 by inserting a finger or the like into the tubular member 20. By moving the tubular member 20 in a direction away from the body surface, the peeling force can be applied toward the center side of the electrode portion 30 from both end sides of the electrode portion 30 in the circumferential direction. As a result, the electrical treatment device can be easily detached, thereby improving convenience.

Note that, while Embodiment 1 describes an exemplary case where the tubular member 20 is provided with the two electrode portions 30, this is not limitative. Two units each including a single tubular member 20 provided with a single electrode portion 30 may be used such that the two units are each connected to the circuit portion 12 of the main body portion 10 through the wiring portion 41. In addition, the number of electrode portions 30 may be 3 or more. In addition, while an exemplary case where a plurality of the electrode portions 30 are disposed side by side in the tubular axis AX direction is described above, this is not limitative, and they may be disposed side by side in the circumferential direction of the tubular member 20.

### Embodiment 2

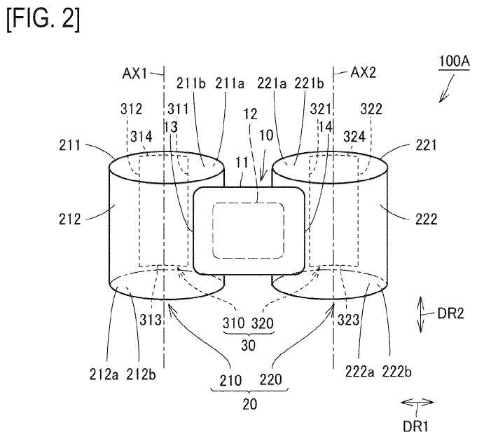

FIG. 2 is a schematic view of an electrical treatment device according to Embodiment 2. With reference to FIG. 2, an electrical treatment device 100A according to Embodiment 2 is described. As illustrated in FIG. 2, the electrical treatment device 100A according to Embodiment 2 includes the main body portion 10, the tubular member 20, and the electrode portion 30.

The main body portion 10 includes the housing case 11 and the circuit portion 12. The housing case 11 has a substantially box shape, and accommodates the circuit portion 12 inside. The housing case 11 includes a first end portion 13 and a second end portion 14 on both sides in a first direction (DR1). The first direction is the direction in which the first end portion 13 and the second end portion 14 are opposite to each other.

The first end portion 13 is located on one side in the first direction, and the second end portion 14 is located on the other side in the first direction. The first end portion 13 and the second end portion 14 substantially linearly extend in plan view. The first end portion 13 and the second end portion 14 extend in the direction orthogonal to the first direction.

The circuit portion 12 is connected to the electrode portion 30 through an electric path (not illustrated in the drawing). Note that, the electric path is provided in each of a first tubular member 210 and a second tubular member 220 described below. The circuit portion 12 controls the current, and voltage and the like flowing through the electrode portion 30. The circuit portion 12 is a circuit board on which a desired circuit is formed.

The tubular member 20 includes the first tubular member 210 and the second tubular member 220. The first tubular member 210 and the second tubular member 220 are disposed with a space therebetween. The arrangement direction in which the first tubular member 210 and the second tubular member 220 are disposed side by side is parallel to the above-mentioned first direction.

The first tubular member 210 is formed in a tubular shape, and has a tubular axis AX1 (first tubular axis). The first tubular member 210 is formed in a circular tubular shape, for example. Note that, the shape of the first tubular member 210 is not limited to the circular tubular shape as long as it is formed in a tubular shape continuously in the circumferential direction while ensuring the region for providing a first electrode portion 310, which will be described below.

The first tubular member 210 includes the first portion 211 that is located on the body surface side of the user in a usage state, and a second portion 212 that is located on the side opposite to the body surface side of the user in a usage state. The first tubular member 210 is formed with the first portion 211 and the second portion 212 that are continuous in the circumferential direction.

The first portion 211 includes an outer surface 211a and an inner surface 211b. The outer surface 211a corresponds to the first surface that faces the body surface side of the user in a usage state. The second portion 212 includes an outer surface 212a and an inner surface 212b. The above-mentioned outer surface 211a and outer surface 212a are continuous in the circumferential direction, and form the outer surface of the first tubular member 210. The inner surface 211b and the inner surface 212b are continuous in the circumferential direction, and form the inner surface of the first tubular member 210.

The second tubular member 220 is formed in a tubular shape, and has a tubular axis AX2 (second tubular axis). The second tubular member 220 is formed in a circular tubular shape, for example. Note that, the shape of the second tubular member 220 is not limited to the circular tubular shape as long as it is formed in a tubular shape continuously in the circumferential direction while ensuring the region for providing a second electrode portion 320, which will be described below.

The second tubular member 220 includes a first portion 221 that is located on the body surface side of the user in a usage state, and a second portion 222 that is located on the side opposite to the body surface side of the user in a usage state. The second tubular member 220 is formed with the first portion 221 and the second portion 222 that are continuous in the circumferential direction.

The first portion 221 includes an outer surface 221a and an inner surface 221b. The outer surface 221a corresponds to the first surface that faces the body surface side of the user in a usage state. The second portion 222 includes an outer surface 222a and an inner surface 222b. The above-mentioned outer surface 221a and outer surface 222a are continuous in the circumferential direction, and form the outer surface of the second tubular member 220. The inner surface 221b and the inner surface 222b are continuous in the circumferential direction, and form the inner surface of the second tubular member 220.

The first tubular member 210 and the second tubular member 220 are disposed such that the tubular axis AX1 and the tubular axis AX2 are orthogonal to the above-mentioned arrangement direction.

The electrode portion 30 is provided on the tubular member 20 such that it is attachable to the body surface of the user. The electrode portion 30 includes the first electrode portion 310 and the second electrode portion 320. The first electrode portion 310 and the second electrode portion 320 may be, for example, a gel pad, or may be a structure including both an adhesive layer and an electrode.

The first electrode portion 310 is provided at the outer surface 211a of the first portion 211 provided in the first tubular member 210. The first electrode portion 310 has a substantially rectangular shape. The first electrode portion 310 includes a first line 311, a second line 312, a third line 313, and a fourth line 314, which make up the outer edge.

The first line 311 and the second line 312 are disposed side by side in the circumferential direction of the first tubular member 210. The first line 311 is located on the side closer to the main body portion 10 than the second line 312. The first line 311 and the second line 312 extend in the direction parallel to the tubular axis AX1. The first line 311 and the second line 312 are approximately parallel to the first end portion 13 of the main body portion 10.

Each of the first line 311 and the second line 312 includes one end portion located on one side in the direction parallel to the tubular axis AX1, and the other end portion located on the other side in the direction parallel to the tubular axis AX1.

The third line 313 connects the above-mentioned one end portions of the first line 311 and the second line 312. The fourth line 314 connects the above-mentioned other end portions of the first line 311 and the second line 312. The third line 313 and the fourth line 314 extend along the circumferential direction of the first tubular member 210.

The second electrode portion 320 is provided at the outer surface 221a of the first portion 221 provided in the second tubular member 220. The second electrode portion 320 has a substantially rectangular shape. The second electrode portion 320 includes a first line 321, a second line 322, a third line 323, and a fourth line 324, which make up the outer edge.

The first line 321 and the second line 322 are disposed side by side in the circumferential direction of the second tubular member 220. The first line 321 is located on the side closer to the main body portion 10 than the second line 322. The first line 321 and the second line 322 extend in the direction parallel to the tubular axis AX2. The first line 321 and the second line 322 are approximately parallel to the second end portion 14 of the main body portion 10.

Each of the first line 321 and the second line 322 includes one end portion located on one side in the direction parallel to the tubular axis AX2, and the other end portion located on the other side in the direction parallel to the tubular axis AX2.

The third line 323 connects the above-mentioned one end portions of the first line 321 and the second line 322. The fourth line 324 connects the above-mentioned other end portions of the first line 321 and the second line 322. The third line 323 and the fourth line 324 extend along the circumferential direction of the second tubular member 220.

The main body portion 10 is fixed to second portions (212, 222) of the tubular member 20 such that at least a part of the main body portion 10 overlaps the second portions (212, 222). More specifically, the main body portion 10 is fixed to the second portions (212, 222) so as to straddle the first tubular member 210 and the second tubular member 220. To be more specific, the first end portion 13 side of the main body portion 10 is fixed to the outer surface 212a of the second portion 212 provided in the first tubular member 210, and the second end portion 14 side of the main body portion 10 is fixed to the outer surface 222a of the second portion 222 provided in the second tubular member 220. Note that, the main body portion 10 may be fixed to the side of the inner surfaces 212b and 222b of the second portions 212 and 222.

In some cases, the electrical treatment device 100A may be mounted to the body surface such that the tubular axis AX1 and the tubular axis AX2 are approximately parallel to the up-down direction (vertical direction), for example. In this case, the gravity of the main body portion 10 acts on the electrical treatment device 100A toward the downward side, that is, one side in the second direction (DR2 direction) orthogonal to the first direction (to be more specific, one side in the direction parallel to the tubular axes AX1 and AX2).

The first electrode portion 310 and the second electrode portion 320 include a conductive gel or an adhesive layer, and buffer the stress from the above-mentioned first direction and the stress from the above-mentioned second direction, for example. In this manner, the first electrode portion 310 and the second electrode portion 320 are less likely to peel off from the body surface.

Further, the first electrode portion 310 includes the first line 311 and the second line 312 that extend approximately parallel to the extending direction of the first end portion 13 of the main body portion 10, and the second electrode portion 320 includes the first line 321 and the second line 322 that extend approximately parallel to the extending direction of the second end portion 14. In this manner, the first electrode portion 310 and the second electrode portion 320 are less likely to peel off, and the electrical treatment device 100A is less likely to peel off from the treatment site during treatment.

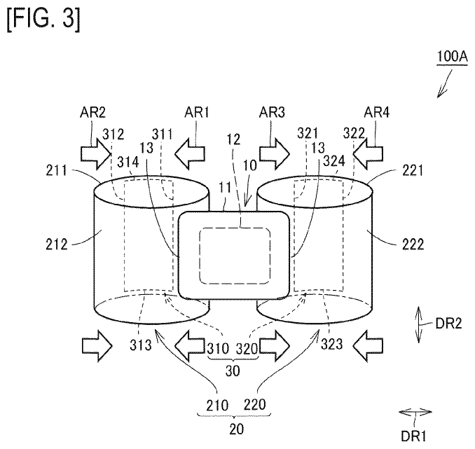

FIG. 3 is a diagram illustrating a state of detaching an electrical treatment device according to Embodiment 2. With reference to FIG. 3, an operation for detaching the electrical treatment device 100A according to Embodiment 2 is described.

As illustrated in FIG. 3, when detaching the electrical treatment device 100A from the body surface, the main body portion 10 is grabbed and moved to the direction away from the body surface. In this manner, the peeling force can be simultaneously applied to the first electrode portion 310 and the second electrode portion 320 on the first tubular member 210 side and on the second tubular member 220.

More specifically, on the first tubular member 210 side, as illustrated with arrows AR1 and AR2 in the drawing, the peeling force toward the inside of the first tubular member 210 in the circumferential direction can be applied to each of both end portions of the first electrode portion 310 in the above-mentioned arrangement direction (to be more specific, the first line 311 and the second line 312).

On the second tubular member 220 side, as illustrated with arrows AR3 and AR4 in the drawing, the peeling force toward the inside of the second tubular member 220 in the circumferential direction can be applied to each of both end portions of the second electrode portion 320 in the above-mentioned arrangement direction (to be more specific, the first line 321 and the second line 322).

In this manner, the first electrode portion 310 and the second electrode portion 320 can be simultaneously peeled from the body surface, and the electrical treatment device 100A can be entirely detached from the body surface. As a result, the electrical treatment device can be easily detached.

Note that, while the above description describes an exemplary case where the electrical treatment device 100A is mounted to the body surface such that the tubular axis AX1 and the tubular axis AX2 are approximately parallel to the up-down direction (vertical direction), the mounting direction of the electrical treatment device 100A is not limited as long as current can be supplied to the body surface through the electrode portion 30. Even in the case where the tubular axis AX1 and the tubular axis AX2 are oriented in a direction different from the up-down direction, the electrical treatment device 100A can be entirely and easily detached from the body surface by moving the main body portion 10 in a direction away from the body surface. The same applies to Embodiments 3 and 4 described below.

### Embodiment 3

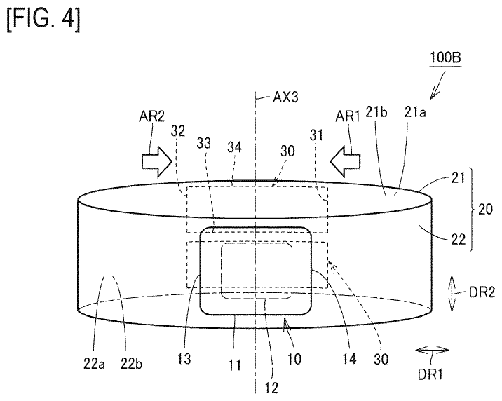

FIG. 4 is a schematic view of an electrical treatment device according to Embodiment 3. With reference to FIG. 4, an electrical treatment device 100B according to Embodiment 3 is described.

As illustrated in FIG. 4, the electrical treatment device 100B according to Embodiment 3 is different from the electrical treatment device 100A according to Embodiment 2 in the configurations of the tubular member 20 and the electrode portion 30.

The tubular member 20 is composed of a single tubular member. The tubular member 20 includes a tubular axis AX3. The tubular axis AX3 is a direction orthogonal to the first direction in which the first end portion 13 and the second end portion 14 of the main body portion 10 are opposite to each other in plan view. The tubular member 20 includes the first portion 21 that is located on the body surface side of the user in a usage state and the second portion 22 that is located on the side opposite to the body surface side of the user. The tubular member 20 is formed with the first portion 21 and the second portion 22 that are continuous in the circumferential direction. The tubular member 20 is provided with the two electrode portions 30.

The first portion 21 includes the outer surface 21a and the inner surface 21b, and the second portion 22 includes the outer surface 22a and inner surface 22b. The outer surface 21a of the first portion 21 corresponds to the first surface that faces the body surface side of the user in a usage state.

Each of the two electrode portions 30 has a substantially rectangular shape. Each electrode portion 30 includes a first line 31, a second line 32, a third line 33, and a fourth line 34, which make up the outer edge. Note that, the first to fourth lines 31 to 34 have substantially the same configurations as those of the first to fourth lines 311 to 314 according to Embodiment 2, and therefore the description thereof will be omitted.

The main body portion 10 is disposed to overlap the electrode portion 30 in plan view. The main body portion 10 is disposed on the opposite side of the electrode portion 30 in the circumferential direction of the tubular member 20. The main body portion 10 is fixed to the second portion 22 such that the main body portion 10 entirely overlaps the second portion 22. The main body portion 10 may be fixed to the outer surface 22a of the second portion 22, or to the inner surface 22b of the second portion 22.

In some cases, the electrical treatment device 100B is mounted to the body surface such that the tubular axis AX3 is approximately parallel to the up-down direction (vertical direction), for example. In this case, the gravity of the main body portion 10 acts on the electrical treatment device 100B toward the downward side, that is, one side in the second direction (DR2 direction) orthogonal to the first direction (to be more specific, one side in the direction parallel to the tubular axis AX3).

Even in this case, as in Embodiment 2, the electrode portion 30 includes the first line 31 and the second line 32 extending approximately parallel to the extending direction of the first end portion 13 of the main body portion 10, with the function of buffering the stress. In this manner, the electrode portion 30 is less likely to peel off, and the electrical treatment device 100B is less likely to peel off from the treatment site during treatment.

When detaching the electrical treatment device 100B from the body surface, the main body portion 10 is grabbed and moved to the direction away from the body surface. In this manner, the peeling force can be applied to the electrode portion 30.

More specifically, as illustrated with arrows AR1 and AR2 in the drawing, the peeling force toward the inside of the tubular member 20 in the circumferential direction can be applied to each of both end portions of the electrode portion 30 in the first direction (to be more specific, the first line 31 and the second line 32).

In this manner, by a single operation of moving the main body portion 10, the electrical treatment device 100B can be entirely and easily detached from the body surface.

### First Modified Example

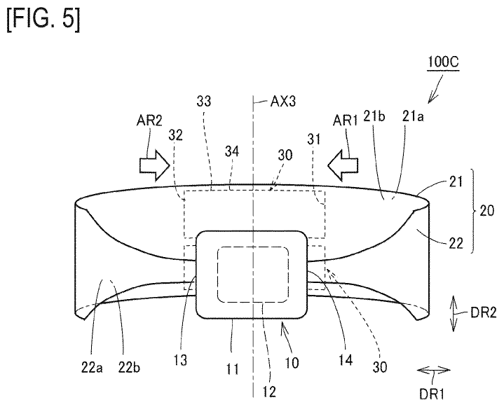

FIG. 5 is a schematic view of an electrical treatment device according to a first modified example. With reference to FIG. 5, an electrical treatment device 100C according to the first modified example is described.

As illustrated in FIG. 5, the electrical treatment device 100C according to the first modified example is different from the electrical treatment device 100B according to Embodiment 3 in the shape of the second portion 22. Other configurations are almost the same.

The length of the first portion 21 in the direction parallel to the tubular axis AX3 direction of the tubular member 20 is constant in the circumferential direction of the tubular member 20. The length of second portion 22 in the direction parallel to the tubular axis AX3 direction partially varies in the above-mentioned circumferential direction. The second portion 22 has a portion where the length in the direction parallel to the tube axial direction is shorter than that of the first portion 21. In the region overlapping the main body portion 10, the length of the second portion 22 in the direction parallel to the tubular axis AX3 direction is shorter than that of the first portion 21. To be more specific, the length of the second portion 22 in the direction parallel to the tubular axis AX3 direction decreases from the boundary with the first portion 21 toward the main body portion 10.

Even with the above-mentioned configuration, the electrical treatment device 100C according to the first modified example achieves substantially the same effect as that of the electrical treatment device 100B according to Embodiment 3. In addition, with the second portion 22 having the above-mentioned shape, the area of the tubular member 20 can be reduced, and the weight and manufacturing cost of the electrical treatment device 100C can be reduced.

Note that, the configuration of the tubular member 20 of the above-mentioned first modified example may be applied to the first tubular member 210 and the second tubular member 220 of Embodiment 2 and Embodiment 4 described below.

### Embodiment 4

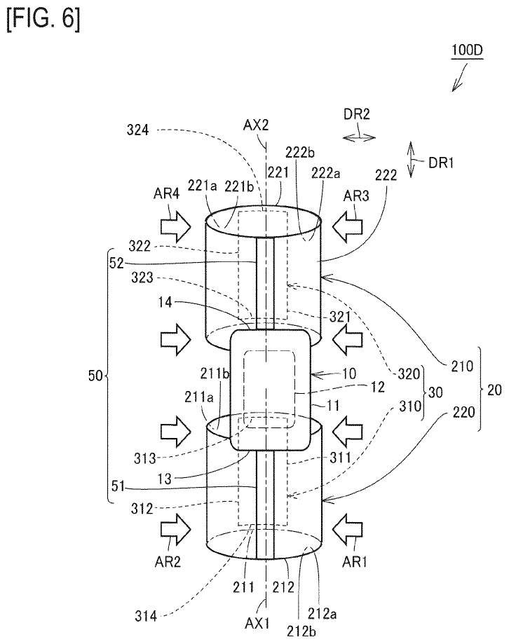

FIG. 6 is a schematic view of an electrical treatment device according to Embodiment 4. With reference to FIG. 6, an electrical treatment device 100D according to Embodiment 4 is described.

As illustrated in FIG. 6, the electrical treatment device 100D according to Embodiment 4 is different from the electrical treatment device 100A according to Embodiment 2 in that a fixing member 50 is provided, and in the orientations of the first tubular member 210 and the second tubular member 220. Other configurations are almost the same.

The fixing member 50 is a member where the main body portion 10 is fixed. The fixing member 50 includes a first fixing portion 51 and a second fixing portion 52.

The first fixing portion 51 is fixed to the second portion 212 provided in the first tubular member 210. To be more specific, the first fixing portion 51 is fixed to the outer surface 212a of the second portion 212. The first fixing portion 51 extends along the tubular axis AX1 direction. The first fixing portion 51 is provided from one end to the other end of the second portion 212 in the direction parallel to the tubular axis AX1 direction, for example.

The second fixing portion 52 is fixed to the second portion 222 provided in the second tubular member 220. To be more specific, the second fixing portion 52 is fixed to the outer surface 222a of the second portion 222. The second fixing portion 52 extends along the tubular axis AX2 direction. The second fixing portion 52 is provided from one end to the other end of the second portion 222 in a direction parallel to the tubular axis AX2 direction, for example. The second fixing portion 52 is disposed on substantially the same straight line as the first fixing portion 51.

The first fixing portion 51 and the second fixing portion 52 are composed of a resin member such as plastic, or a metal member. The first fixing portion 51 and the second fixing portion 52 may be provided in a rod shape, or a flat shape.

In plan view, the tubular axis AX1 of the first tubular member 210 and the tubular axis AX2 of the second tubular member 220 are parallel to the arrangement direction in which the first tubular member 210 and the second tubular member 220 are disposed side by side. In addition, the tubular axis AX1 of the first tubular member 210 and the tubular axis AX2 of the second tubular member 220 are parallel to the first direction (DR1 direction) in which the first end portion 13 and the second end portion 14 of the main body portion 10 are opposite to each other.

In this case, on the first electrode portion 310 side, the first line 311 and the second line 312 are disposed side by side in the circumferential direction of the first tubular member 210. The third line 313 connects the end portions of the first line 311 and the second line 312 located on the side closer to the main body portion 10 in the direction parallel to the tubular axis AX1. The fourth line 314 connects the end portions of the first line 311 and the second line 312 located on the side remote from the main body portion 10 in the direction parallel to the tubular axis AX1.

Likewise, on the second electrode portion 320 side, the first line 321 and the second line 322 are disposed side by side in the circumferential direction of the second tubular member 220. The third line 323 connects the end portions of the first line 321 and the second line 322 located on the side closer to the main body portion 10 in the direction parallel to the tubular axis AX2. The fourth line 324 connects the end portions of the first line 321 and the second line 322 located on the side remote from the main body portion 10 in the direction parallel to the tubular axis AX2.

The above-mentioned third lines 313 and 323 and the above-mentioned fourth lines 314 and 324 are approximately parallel to the first end portion 13 and the second end portion 14 of the main body portion 10 in the state where the first electrode portion 310 and the second electrode portion 320 are attached, or a state as viewed in plan view.

The main body portion 10 is fixed to the fixing member 50. More specifically, in the arrangement direction in which the first tubular member 210 and second tubular member 220 are disposed side by side, one end side of the main body portion 10 is fixed to the first fixing portion 51, and the other end side of the main body portion 10 in the arrangement direction is fixed to the second fixing portion 52. Specifically, the first end portion 13 of the main body portion 10 is fixed to the first fixing portion 51, and the second end portion 14 of the main body portion 10 is fixed to the second fixing portion 52. In this manner, the main body portion 10 is fixed to the first tubular member 210 and the second tubular member 220 through the fixing member 50.

In some cases, the electrical treatment device 100D is mounted to the body surface such that the tubular axes AX1 and AX2 are approximately parallel to the up-down direction (vertical direction). In this case, the gravity of the main body portion 10 acts on the electrical treatment device 100D toward the downward side (one side of the tube axial direction).

Also in Embodiment 4, the first electrode portion 310 and the second electrode portion 320 serve the function of buffering the stress. Additionally, with the tensile force of the first line 311, the second line 312 and the first line 321, the second line 322 extending in directions parallel to the tubular axes AX1 and AX2, peeling in the DR1 direction is less likely to occur, and the electrical treatment device 100D less likely to peel off from the treatment site during treatment.

When detaching the electrical treatment device 100D from the body surface, the main body portion 10 is grabbed and moved to the direction away from the body surface. At this time, the fixing member 50 where the main body portion 10 is fixed (to be more specific, the first fixing portion 51 and the second fixing portion 52) also moves, and as a result, the peeling force can be simultaneously applied to the first electrode portion 310 and the second electrode portion 320.

More specifically, as illustrated with arrows AR1 and AR2 in the drawing, the peeling force toward the inside of the first tubular member 210 in the circumferential direction can be applied to each of both end portions of the first electrode portion 310 (to be more specific, the first line 311 and the second line 312) in the circumferential direction of the first tubular member 210.

In addition, as illustrated with arrows AR3 and AR4 in the drawing, the peeling force toward the inside of the second tubular member 220 in the circumferential direction can be applied to each of both end portions of the second electrode portion 320 in the circumferential direction of the second tubular member 220 (to be more specific, the first line 321 and the second line 322).

Further, the first fixing portion 51 is provided from the one end to the other end of the first tubular member 210 in the tubular axis AX1 direction, and the second fixing portion 52 is provided from the one end to the other end of the second tubular member 220 in the tubular axis AX2 direction. Thus, the peeling force can be uniformly applied to the first electrode portion 310 and the second electrode portion 320.

In this manner, also in Embodiment 4, by a single operation of moving the main body portion 10, the electrical treatment device 100D can be entirely and easily detached from the body surface.

Note that, while the above-described Embodiments 2 to 4 and modified examples describe an exemplary case where the first electrode portion 310, the second electrode portion 320, and the electrode portion 30 have rectangular shapes, this is not limitative, and may have any appropriate shape such as a circular shape, an elliptical shape, a polygonal shape, or an oval shape. In addition, the line making up the outer edge of the electrode portion is not limited to the straight line, and may be zigzag or curved.

### Supplementary Notes

As described above, the present embodiment includes the following disclosures.

Configuration 1: An electrical treatment device (100, 100A, 100B, 100C, 100D) including:
a tubular member (20); and
an electrode portion (30) provided at the tubular member (20) such that the electrode portion (30) is attachable to a body surface of a user, in which
the tubular member (20) includes a first portion (21) located on the body surface side of the user in a usage state and a second portion (22) located on a side opposite to the body surface side of the user,
the first portion (21) includes a first surface that faces the body surface side of the user in a usage state, and
the electrode portion (30) is provided at the first surface.

Configuration 2: The electrical treatment device (100A, 100B, 100C, 100D) according to configuration 1, further including a main body portion (10) fixed to the tubular member (20), in which
the main body portion (10) is fixed to the second portion (22) such that at least a part of the main body portion (10) overlaps the second portion (22).

Configuration 3: The electrical treatment device (100A) according to configuration 2, in which
the tubular member (20) includes a first tubular member (210) and a second tubular member (220) disposed with a space between the first tubular member (210) and the second tubular member (220),
each of the first tubular member (210) and the second tubular member (220) includes the first portion (211, 221) and the second portion (212, 222),
the electrode portion (30) includes a first electrode portion (310) provided at the first surface provided in the first tubular member (210), and a second electrode portion (320) provided at the first surface provided in the second tubular member (220), and the main body portion (10) is fixed to the second portion (212) provided in the first tubular member (210) and the second portion (222) provided in the second tubular member (220) so as to straddle the first tubular member (210) and the second tubular member (220).

Configuration 4: The electrical treatment device (100A) according to configuration 3, in which
a first tubular axis provided in the first tubular member (210) and a second tubular axis provided in the second tubular member (220) are parallel to each other, and
the first tubular axis and the second tubular axis are orthogonal to a direction in which the first tubular member (210) and the second tubular member (220) are disposed side by side in plan view.

Configuration 5: The electrical treatment device (100D) according to configuration 2, in which
the tubular member (20) includes a first tubular member (210) and a second tubular member (220) disposed with a space between the first tubular member (210) and the second tubular member (220),
each of the first tubular member (210) and the second tubular member (220) includes the first portion (211, 221) and the second portion (212, 222),
the electrode portion (30) includes a first electrode portion (310) provided at the first surface provided in the first tubular member (210), and a second electrode portion (320) provided at the first surface provided in the second tubular member (220),
a fixing member (50) fixed to the main body portion (10) is further provided,
the fixing member (50) includes a first fixing portion (51) fixed to the second portion (212) provided in the first tubular member (210), and a second fixing portion (52) fixed to the second portion (222) provided in the second tubular member (220), and one end side of the main body portion (10) in an arrangement direction in which the first tubular member (210) and the second tubular member (220) are disposed side by side is fixed to the first fixing portion (51), and another end side of the main body portion (10) in the arrangement direction is fixed to the second fixing portion (52), and thus the main body portion (10) is fixed to the first tubular member (210) and the second tubular member (220) through the fixing member (50).

Configuration 6: The electrical treatment device (100D) according to configuration 5, in which a first tubular axis provided in the first tubular member (210) and a second tubular axis provided in the second tubular member (220) are parallel to the arrangement direction in plan view.

Configuration 7: The electrical treatment device (100C) according to any one of configurations 1 to 6, in which
a length of the first portion (21) in a direction parallel to a tube axial direction of the tubular member (20) is constant in a circumferential direction, and
the second portion (22) includes a portion where a length in the direction parallel to the tube axial direction is smaller than that of the first portion (21) in the region overlapping the main body portion (10).

It is to be understood that the embodiments and modified examples disclosed herein are illustrative in all respects and are not to be construed as limiting. The scope of the present invention is defined by the claims, and all modified examples within the meaning and scope equivalent to the claims are intended to be included therein.

### REFERENCE NUMERALS LIST

10 Main Body Portion
11 Housing case
12 Circuit portion
13 First End Portion
14 Second End Portion
20 Tubular Member
21 First Portion
21a Outer Surface
21b Inner Surface
22 Second Portion
22a Outer Surface
22b Inner Surface
30 Electrode Portion
31 First Line
32 Second Line
33 Third Line
34 Fourth Line
41 Wiring portion
50 Fixing Member
51 First Fixing Portion
52 Second Fixing Portion
100, 100A, 100B, 100C, 100D Electrical Treatment Device
210 First Tubular Member
211 First Portion
211a Outer Surface
211b Inner Surface
212 Second Portion
212a Outer Surface
212b Inner Surface
220 Second Tubular Member
221 First Portion
221a Outer Surface
221b Inner Surface
222 Second Portion
222a Outer Surface
222b Inner Surface
310 First Electrode Portion
311 First Line
312 Second Line
313 Third Line
314 Fourth Line
320 Second Electrode Portion
321 First Line
322 Second Line
323 Third Line
324 Fourth Line
AX1, AX2, AX3 Tubular Axis

What is claimed is:

1. An electrical treatment device comprising:
a tubular member having both ends opened in a tube axial direction; and
an electrode portion provided at the tubular member such that the electrode portion is attachable to a body surface of a user, wherein:
the tubular member includes a first portion located on the body surface side of the user in a usage state and a second portion located on a side opposite to the body surface side of the user;
the first portion includes an inner surface and an outer surface as a first surface that faces the body surface side of the user in a usage state;
the second portion includes an inner surface and an outer surface;
the outer surfaces of the first surface and the outer surface of the second portion are continuous in a circumferential direction, and form the outer surface of the tubular member; and,
the electrode portion is provided at the first surface.

2. The electrical treatment device according to claim 1, further comprising:
a main body portion fixed to the tubular member, wherein:
the main body portion is fixed to the second portion such that at least a part of the main body portion overlaps the second portion.

3. The electrical treatment device according to claim 2, wherein:
the tubular member includes a first tubular member and a second tubular member disposed with a space between the first tubular member and the second tubular member;
each of the first tubular member and the second tubular member includes the first portion and the second portion;
the electrode portion includes a first electrode portion provided at the first surface provided in the first tubular member, and a second electrode portion provided at the first surface provided in the second tubular member; and,
the main body portion is fixed to the second portion provided in the first tubular member and the second portion provided in the second tubular member so as to straddle the first tubular member and the second tubular member.

4. The electrical treatment device according to claim 3, wherein:
a first tubular axis provided in the first tubular member and a second tubular axis provided in the second tubular member are parallel to each other; and,
the first tubular axis and the second tubular axis are orthogonal to a direction in which the first tubular member and the second tubular member are disposed side by side in plan view.

5. The electrical treatment device according to claim 2, wherein:
the tubular member includes a first tubular member and a second tubular member disposed with a space between the first tubular member and the second tubular member;
each of the first tubular member and the second tubular member includes the first portion and the second portion;
the electrode portion includes a first electrode portion provided at the first surface provided in the first tubular member, and a second electrode portion provided at the first surface provided in the second tubular member;
a fixing member fixed to the main body portion is further provided;
the fixing member includes a first fixing portion fixed to the second portion provided in the first tubular member, and a second fixing portion fixed to the second portion provided in the second tubular member; and,
one end side of the main body portion in an arrangement direction in which the first tubular member and the second tubular member are disposed side by side is fixed to the first fixing portion, and another end side of the main body portion in the arrangement direction is fixed to the second fixing portion, and thus the main body portion is fixed to the first tubular member and the second tubular member through the fixing member.

6. The electrical treatment device according to claim 5, wherein:
a first tubular axis provided in the first tubular member and a second tubular axis provided in the second tubular member are parallel to the arrangement direction in plan view.

7. The electrical treatment device according to claim 1, wherein:
a length of the first portion in a direction parallel to a tube axial direction of the tubular member is constant in a circumferential direction; and,
the second portion includes a portion where a length in the direction parallel to the tube axial direction is smaller than the length of the first portion.

8. The electrical treatment device according to claim 2, wherein:
a length of the first portion in a direction parallel to a tube axial direction of the tubular member is constant in a circumferential direction; and,
the second portion includes a portion where a length in the direction parallel to the tube axial direction is smaller than the length of the first portion.

9. The electrical treatment device according to claim 3, wherein:
a length of the first portion in a direction parallel to a tube axial direction of the tubular member is constant in a circumferential direction; and,
the second portion includes a portion where a length in the direction parallel to the tube axial direction is smaller than the length of the first portion.

10. The electrical treatment device according to claim 4, wherein:
a length of the first portion in a direction parallel to a tube axial direction of the tubular member is constant in a circumferential direction; and,
the second portion includes a portion where a length in the direction parallel to the tube axial direction is smaller than the length of the first portion.

11. The electrical treatment device according to claim 5, wherein:
a length of the first portion in a direction parallel to a tube axial direction of the tubular member is constant in a circumferential direction; and,
the second portion includes a portion where a length in the direction parallel to the tube axial direction is smaller than the length of the first portion.

12. The electrical treatment device according to claim 6, wherein:
a length of the first portion in a direction parallel to a tube axial direction of the tubular member is constant in a circumferential direction; and,
the second portion includes a portion where a length in the direction parallel to the tube axial direction is smaller than the length of the first portion.
